(19) United States
(12) Patent Application Publication
(10) Pub. No.: US 2005/0058678 A1
(43) Pub. Date: Mar. 17, 2005

(54) Two-coat cosmetic product, uses thereof and makeup kit comprising the same

(76) Inventors: Audrey Ricard, Paris (FR); Helene Khachikian, Alfortville (FR); Patricia Lemann, Creteil (FR); Franck Girier Dufournier, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/903,037

(22) Filed: Aug. 2, 2004

Related U.S. Application Data
(60) Provisional application No. 60/493,346, filed on Aug. 8, 2003.

(30) Foreign Application Priority Data
Aug. 1, 2003 (FR) 03 09563

(51) Int. Cl. A61K 7/06; A61K 7/11

(52) U.S. Cl. 424/401; 424/70.12

(57) ABSTRACT

The disclosure relates to a cosmetic makeup product and/or care product comprising at least one first and at least one second composition, the at least one first composition comprising a first physiologically acceptable medium and having a haze index, (Th−Td)/Th*100, of greater than or equal to 40%, and a transparency index, Th, of greater than or equal to 70%, and the at least one second composition comprising a second physiologically acceptable medium. The disclosure also relates to a makeup process and to a makeup kit comprising the same.

This application claims benefit of U.S. Provisional Application No. 60/493,346, filed Aug. 8, 2003.

The present disclosure relates to cosmetic products comprising at least one first and at least one second composition that may be applied successively either to human facial or body skin, to the human lower and upper eyelids, to the lips and to integuments, for instance the nails, the eyebrows, the eyelashes or the hair, and also to a two-coat makeup process for the human face and body.

Each composition can be in a form chosen from a free or compacted powder, a foundation, a makeup rouge, an eyeshadow, a concealer product, a blusher, a lipstick, a lip balm, a lip gloss, a lip or eye pencil, a mascara, an eyeliner, a nail varnish or a body-makeup or a skin-coloring product.

The at least one first composition may reduce or eliminate skin defects via an optical effect. It may allow imperfections on the surface of the skin, for example, wrinkles, pores, marks, irregular textures, differences in tone or spots, to be visually masked. It also may form a translucent film on the skin and thus, give the skin a natural appearance while at the same time improving its uniformity and radiance.

The at least one first composition of the disclosure may not leave any visible traces of white or tinted color on the skin. The consumer does not need to choose a tint of product that is closest to the consumer's flesh tint, since. the composition of the disclosure may adapt to most types of skin and may produce an immediate visual effect of enhancement of the uniformity of the skin by masking its defects.

The at least one first composition also may make it possible to modify the visual perception of the volume of the part of.the body onto which it is applied.

The at least one first composition may be used as a preparatory base for smoothing the skin, such as the lips and their contour, via an optical effect. When combined with the at least one second composition of the present disclosure, the at least one first composition may improve the staying power and reduce the migration of the at least one second composition.

In addition, the selection of at least one second glossy composition that modifies the perception of the volume of the part of the body onto which the composition is applied may make it possible to obtain a cosmetic product, for instance a makeup product, that may have good staying power, may be glossy, comfortable, and may reduce migration.

Accordingly, the present disclosure relates to a cosmetic makeup and/or care product with improved staying power and with reduced migration. This makeup product may mask imperfections of the skin, for instance of the lips and the contour of the lips. Specifically, the at least one first composition may make it possible to improve the staying power and to reduce the migration of the at least one second composition.

Another aspect of the present disclosure is a makeup product that is not only glossy but also may have good staying power and may not migrate.

It is a known technique to increase the proportion of the oily phase at the expense of the particulate phase in order to increase the gloss of a makeup or care product, dispersing, as best as possible, the particulate phase. This technique may lead to a film that is not uniform on the lips, giving an unattractive appearance because of poor dispersion of the pigmentary particles. Furthermore, an excessively small amount of pigments often leads to a film that covers poorly. In addition, the gloss of the composition may have a tendency to decrease over time, for instance, because of the poor staying power of the film over time.

It is also a known practice to thicken the oils with polymeric thickeners, for example, polyolefins. Unfortunately, these known oil thickeners must be used in large amounts in order to obtain efficient thickening. An excessively large amount of thickener, however, may give the composition, insufficient cosmetic properties, such as a tacky feel and a lack of glide. These drawbacks can being very irritating, or even unacceptable to some consumers.

A need therefore remains to reduce the large migration of a liquid fatty phase, charged with dyestuffs, that leads to an unattractive effect around the lips, which, for example, accentuates the wrinkles and fine lines. Consumers often mention this migration as being a major defect of standard lipsticks. Accordingly, the cosmetic composition of the disclosure may make it possible. to limit the migration of this phase into the wrinkles and fine lines, once it has been deposited on the skin or the lips; such a property may be desirable for a lipstick.

The product of the present disclosure is also covering, in the sense that it masks, the imperfections of the skin, such as of the lips and the contour of the lips.

Accordingly, the present disclosure relates to the combining of at least one first and at least one second composition, wherein the at least one first composition comprises a first physiologically acceptable medium and having a haze index, (Th−Td)/Th*100, of greater than or equal to 40% and a transparency index, Th, of greater than or equal to 70%, and the at least one second composition comprises a second physiologically acceptable medium, which makes it possible to obtain a two-coat makeup that reduces migration, has good staying power and/or is comfortable when applied and over time (i.e., does not dry out and/or does not cause tautness).

For instance, the product of the present disclosure may allow the production of glossy, non-migrating deposits with improved staying power. These deposits do not form whitish traces on the skin, are translucent, and allow the skin.to be made up, while at the same time preserve its color and its natural appearance. The product of the disclosure, thus, may allow a homogeneous and attractive makeup result.

These advantageous properties may make the product of the disclosure suitable for producing makeup products for the lips, such as lipsticks and lip glosses; for the eyes, such as mascara, eyeliners or eyeshadows; or for facial skin, such as foundations. It also may make it possible to produce body makeup products for diminishing the appearance of scars or skin structure defects.

One aspect of the present disclosure is thus a cosmetic product, such as a makeup and/or care product, comprising at least one first and at least one second composition, the at least one first composition comprising a first physiologically acceptable medium and having a haze index, (Th−Td)/Th*100, of greater than or equal to 40%, and a transparency index, Th, of greater than or equal to 70%, and the at least one second composition comprising a second physiologically acceptable medium.

As used herein, the term “makeup product” means a product comprising a coloring agent that allows for the deposit of a color onto a keratin material (e.g., the skin, the lips, or integuments), such as lipsticks, makeup powders, eyeliners, foundations, self-tanning products, or semi-permanent makeup products (e.g., tattoos).

The product according to the present disclosure comprises at least two physiologically acceptable compositions packaged separately or together in the same packaging article or in at least two separate or distinct packaging articles.

For instance, these compositions may be packaged separately such as, in separate or distinct packaging articles.

One aspect of the present disclosure is thus, for instance, a cosmetic makeup or care product in a form chosen from a foundation, a makeup rouge, an eyeshadow, a lipstick, a product having, for example, care properties, an eyeliner, a concealer product and a body makeup product (such as a tattoo).

A further aspect of the present disclosure is a makeup kit comprising a cosmetic makeup or care product as defined above, wherein the various compositions are packaged separately and are accompanied by suitable applicators. The applicator may be chosen from soft-bristled brushes, hard-bristled brushes, pens, pencils, felts, quills, sponges, and foams.

The at least one first composition of the product according to the present disclosure may become a basecoat once applied to a keratin material, and the at least one second composition may become a topcoat. However, it is possible to apply, under the at least one first composition, an undercoat that may or may not have the composition of the second coat.

It is also possible to deposit an overcoat onto the at least one second composition, which may be identical or different, to that of the at least one first composition. Thus, the makeup obtained may be a two-coat makeup.

According to the present disclosure, for example, the basecoat may be chosen from a makeup base, a correcting product, a foundation, a makeup powder, a lipstick, an eyeliner and a body makeup product, and the topcoat may be chosen from a protective and care product and a lip gloss.

The present disclosure also relates to a process for making up the skin and/or the lips and/or integuments comprising applying a cosmetic makeup product as defined above to the skin and/or the lips and/or integuments.

Another aspect of the present disclosure is also a makeup or care process for human skin and/or lips and/or integuments comprising applying to the skin, the lips and/or integuments a first coat of at least one first composition comprising a physiologically acceptable medium and having a haze index, (Th−Td)/Th*100, of greater than or equal to 40% and a transparency index, Th, of greater than or equal to 70%, and then applying a second coat of at least one second composition comprising a second physiologically acceptable medium.

The second coat may form patterns and may be applied with a pen, a pencil or any other instrument (e.g., sponge, finger, soft-bristled brush, hard-bristled brush, foam, quill, etc.). This makeup may also be applied to makeup accessories, for instance, false nails, false eyelashes or wigs, or alternatively to pastilles or patches adhering to the skin or the lips (such as beauty spots).

Yet another aspect of the present disclosure is a make-up support comprising a first coat of at least one first composition comprising a first physiologically acceptable medium and having a haze index, (Th−Td)/Th*100, of greater than or equal to 40% and a transparency index, Th, of greater than or equal to 70%, and a second coat of at least one second composition comprising a second physiologically acceptable medium, deposited onto all or part of the first coat.

This support may be, for instance, a hairpiece such as a wig, false nails or false eyelashes, or alternatively, patches adhering to the skin or the lips (such as beauty spots).

Still another aspect of the present disclosure is the use of a cosmetic makeup product comprising at least one first and at least one second composition, the at least one first composition comprising a first physiologically acceptable medium and having a haze index, (Th−Td)/Th*100, of greater than or equal to 40% and a transparency index, Th, of greater than or equal to 70%, and the at least one second composition comprising a second physiologically acceptable medium, wherein the product left on the skin and/or the lips and/or the integuments results in a glossy, comfortable deposit with good staying power, good color fastness and good gloss fastness that does not migrate and/or which masks the imperfections of the skin, the lips or the nails.

First Composition

The at least one first composition according to the present disclosure thus comprises a first physiologically acceptable medium.

As used herein, the term “physiologically acceptable medium” means a non-toxic medium that may be applied to human skin, integuments, lips, or the face.

As used herein, the term “cosmetically acceptable” means a composition of pleasant appearance, odor, taste, and feel.

The at least one first composition according to the present disclosure comprises a first physiologically acceptable medium and has a haze index, (Th−Td)/Th*100, of greater than or equal to 40% and a transparency index, Th, of greater than or equal to 70%.

Such a composition can give the complexion, the lips and the skin in general, greater visual uniformity, greater homogeneity, transparency and radiance. When applied to the lips, the at least one first composition of the present disclosure may allow the lips to be made smooth by an optical effect, and may allow the wrinkles and imperfections of the lips and the contour of the lips to be masked.

The haze index, (Th−Td)/Th*100, may be measured according to the protocol described below using a spectrophotometer and an integration sphere, for example placed behind the sample.
Th is the hemispheric transmittance of the composition: it is the ratio of the light intensity received by the composition to the light intensity released by the composition in all directions,
Td is the direct transmittance of the composition: it is the ratio of the light intensity received by the composition to the light intensity released by the composition in the same axis.

The Th and Td values may be measured using a Varian Cary 300 spectrophotometer and a Labsphere brand integration sphere placed behind the sample based on the following protocol. A 20 μm thick film of the at least one first composition according to the present disclosure is spread onto a quartz crucible slide and then placed at 37° C. for 5 minutes.

To measure the Th value, the spectrophotometer is used in diffuse transmission mode, at a wavelength ranging from 400 nm to 700 nm. The machine is set in % T transmission mode, at a scanning speed of 240 nm/min, in “double reverse” mode. A baseline correction is made by measuring a reference empty slide. This gives the maximum value of the intensity that may be transmitted. The quartz slide comprising the film of the at least one first composition is placed in the measuring compartment and the Th value is measured.

The Td value may be measured using the same spectrophotometer, according to the following protocol. The spectrophotometer is used in direct transmission mode, at a wavelength ranging from 400 nm to 700 nm. The machine is set in % T transmission mode, at a scanning speed of 240 nm/min, in double mode. An empty quartz slide is placed in the reference compartment and the quartz slide containing the sample is placed in the measuring compartment. The Td is measured.

The values of Th and Td are the means of each of the spectral values measured.

A higher value of the haze index, (Th−Td)/Th*100, indicates a better haze effect. A higher Td value indicates the more transparent the composition.

As disclosed herein, the haze index of the at least one first composition is greater than or equal to 40% and for example, greater than or equal to 50%.

The transparency index may, for example, be greater than or equal to 85% such as, greater than or equal to 95%.

As disclosed herein, the at least one first composition comprises a compound chosen from mineral and organic compounds capable of giving the composition such a haze index and such a transparency index. This compound is, for example, present in an amount that is sufficient to give the composition the haze index and the transparency index defined above.

The compound may be chosen, for example, from fillers, organopolysiloxanes, and non-silicone polymers of low molecular mass.

As used herein, the term “fillers” means white or colored, mineral or organic, lamellar or non-lamellar particles, which are solid at room temperature (25° C.) and atmospheric pressure, which do not interact chemically with the other ingredients of the composition and which are, for example, insoluble in these ingredients, even when these ingredients are brought to a temperature above room temperature.

As used herein, the term “organic fillers” means fillers comprising a single organic chemical compound or fillers comprising an organic substrate coated with another organic or mineral compound.

As used herein, the term “mineral fillers” means fillers comprising a single mineral chemical compound or fillers comprising a mineral substrate coated with another organic or mineral compound.

The fillers may be used to give the composition of the disclosure a sufficient haze index and a sufficient transparency index, and may be, for example, in the form of a powder. The mean particle size of the fillers can be, for example, less than or equal to 50 microns, for example 30 microns, such as 15 microns and further, for example, less than or equal to 10 microns, such as from 3 microns to 5 microns.

Among the fillers of natural or synthetic origin that may be used, non-limiting mention may be made of fillers chosen from talc, starch, mica, silica, Nylon powders, polyethylene powders, poly-p-alanine, and poly(methyl methacrylates).

The organosiloxanes may be in the form of a gel comprising an elastomeric organopolysiloxane of three-dimensional structure, included in at least one hydrocarbon-based oil and/or one silicone oil.

The elastomeric organopolysiloxanes may be chosen from the crosslinked polymers described in European Patent Application EP-A-0 295 886.

According to EP-A-0 295 886, the elastomeric organopolysiloxanes may be obtained by an addition reaction and crosslinking of at least:
(a) an organopolysiloxane comprising at least two lower alkenyl groups per molecule;
(b) an organopolysiloxane comprising at least two hydrogen atoms linked to one silicon atom per molecule; and
(c) a platinum-type catalyst.

The elastomeric organopolysiloxanes may also be chosen from those described in U.S. Pat. No. 5,266,321. According to U.S. Pat. No. 5,266,321, the elastomeric organopolysiloxanes may be chosen, for example, from:
i) polyorganopolysiloxanes comprising at least one unit chosen from R₂SiO and RSiO_{1.5} and optionally at least one unit chosen from R₃SiO_{0.5} and SiO₂ wherein R, R₂ and R₃, which may be identical or different, are chosen from hydrogen atoms; alkyl groups such as, methyl, ethyl and propyl; aryl groups such as, phenyl and tolyl; and unsaturated aliphatic groups such as, vinyl, and wherein the weight ratio of R₂SiO to RSiO_{1.5} ranges from 1/1 to 30/1;
ii) polyorganopolysiloxanes that are insoluble and swellable in silicone oil, obtained by addition of an organohydrogenopolysiloxane and an organopolysiloxane comprising unsaturated aliphatic groups, such that the amount of hydrogen in the organohydrogenopolysiloxane and of unsaturated aliphatic groups in the organopolysiloxane, respectively, ranges from 1 mol % to 20 mol % when the organopolysiloxane is non-cyclic and ranges from 1 mol % to 50 mol % when the organopolysiloxane is cyclic.

The fatty phase associated with the solid elastomeric organopolysiloxane forms a matting agent comprising at least one oil chosen from hydrocarbon-based oils and silicone oils.

The hydrocarbon-based oils used in combination with the elastomeric organopolysiloxane may be chosen from, but are not limited to, the following:
oils of animal origin such as perhydrosqualene;
plant oils such as liquid triglycerides, for example sunflower oil, maize oil, soybean oil, marrow oil, grapeseed oil, sesame seed oil, hazelnut oil, apricot oil, macadamia oil, castor oil, caprylic/capric acid triglycerides, for instance those sold by the company Stéarineries Dubois or those sold under the names MIGLYOL 810, 812 and 818 by the company Dynamit Nobel;
oils of formula R₉COOR₁₀ wherein R₉ is chosen from higher fatty acid residues comprising from 7 to 19 carbon atoms and R₁₀ is chosen from branched hydrocarbon-based chains comprising from 3 to 20 carbon atoms, for example purcellin oil;
linear and branched hydrocarbons, of mineral or synthetic origin, such as paraffin oils and derivatives thereof, isoparaffin, isohexadecane, squalane and equivalents, such as polyisobutenes and polydecenes;
synthetic esters and ethers; and
mixtures thereof.

The silicone oils that may be used in combination with the elastomeric organopolysiloxane are, for example, chosen from, but are not limited to, the following: linear polysiloxanes that are liquid or pasty at room temperature, such as methylpolysiloxane, methylphenylpolysiloxane, ethylpolysiloxane, ethylmethylpolysiloxane, ethylphenylpolysiloxane, hydroxymethylpolysiloxane and alkylpolydimethylsiloxane; cyclic polysiloxanes such as octamethylcyclopentasiloxane and decamethylcyclopentasiloxane; and mixtures thereof.

In one embodiment, the organopolysiloxane may be present in the organopolysiloxane/fatty phase mixture to form the matting agent in the form of a homogeneous gel in an amount ranging from 3% to 80% by weight.

The organopolysiloxanes known under the commercial references KSG (KSG 6, 16, 17 and 18) from the company Shin Etsu, TREFILS from the company Dow Corning or GRANSILS from the company Grant Industries may be used.

The non-silicone polymers of low molecular mass are chosen from, for example, amide, urethane, and urea polymers. These polymers, for example, may have pendent and/or terminal alkyl or alkylene chains, such as comprising from 8 to 60 carbon atoms for example, from 12 to 40 carbon atoms.

Their weight-average molecular mass ranges from, for example, 200 g/mol to 1000 g/mol.

In some embodiments, the polymeric compound may be a polyamide. For example, the compound may be a polyamide that results from the amidation of a triglyceride with a diamine, optionally in the presence of a C₁₂ to C₄₀ monocarboxylic acid. The triglyceride is, for example, a hydroxylated fatty acid triglyceride comprising from 12 to 30 carbon atoms, for instance ricinoleic acid (or castor oil) and the monocarboxylic acid may be 12-hydroxystearic acid. The diamine may be, for example, ethylenediamine.

According to further aspects of the present disclosure, the at least one first composition comprises at least one fatty phase comprising at least one volatile oil.

As used herein, the term “volatile oil” means any non-aqueous medium capable of evaporating on contact with the skin, at room temperature (25° C.) and atmospheric pressure (760 mmHg). Volatile cosmetic oils, which are liquid at room temperature, for instance have a vapor pressure, measured at room temperature and atmospheric pressure, ranging from 10⁻³ mmHg to 300 mmHg (0.266 Pa to 40000 Pa), such as from 0.02 mmHg to 300 mmHg (2.66 Pa to 40000 Pa) and further, for example, ranging from 0.1 mmHg to 90 mmHg (13 Pa to 12000 Pa).

According to the present disclosure, these volatile oils may be chosen from hydrocarbon-based oils, silicone oils optionally comprising alkyl and alkoxy groups that are pendent or at the end of a silicone chain, and mixtures thereof.

For instance, the volatile oils may be cosmetic oils chosen from oils that do not have a flashpoint, oils with a flashpoint ranging from 40° C. to 100° C., and mixtures thereof, in order to facilitate their use. In addition, the volatile oils, for example, may have a boiling point at atmospheric pressure of less than 220° C. and, for instance, less than 210° C., such as ranging from 110° C. to 210° C. For example, the volatile oils may not be monoalcohols comprising at least 7 carbon atoms.

Examples of volatile oils that may be used in the disclosure include linear and cyclic silicone oils with a viscosity at room temperature of less than 8 cSt and, for instance, comprising from 2 to 7 silicon atoms, these silicones optionally comprise a group chosen from alkyl and alkoxy groups comprising from 1 to 10 carbon atoms. Examples of volatile silicone oils that may be used in the disclosure are chosen from octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

Further examples of volatile oils that may be used in the disclosure include hydrocarbon-based volatile oils comprising from 8 to 16 carbon atoms, and mixtures thereof, and, for example, branched C₈-C₁₆ alkanes, for instance C₈-C₁₆ isoalkanes (also known as isoparaffins), isododecane, isodecane and isohexadecane, for example, the oils sold under the trade names Isopars or Permetyls, and branched C₈-C₁₆ esters, for instance isohexyl neopentanoate, and mixtures thereof.

In some embodiments, the volatile oils of isododecane (Permetyls 99 A), C₈-C₁₆ isoparaffins, for instance Isopar L, E, G or H, and mixtures thereof, optionally combined with decamethyltetrasiloxane or with cyclopentasiloxane, are used.

Volatile fluoro oils may also be used.

These volatile oils may be present in an amount ranging from 5% to 80% and for example, ranging from 10% to 30%, by weight relative to the total weight of the composition.

According to another aspect of the present disclosure, the at least one first composition comprises at least one organic filler and at least one mineral filler. The organic filler and the mineral filler may have, independently of one another, a mean particle size of less than or equal to 50 microns, for example 30 microns, such as 15 microns and further, for example, less than or equal to 10 microns, such as from 3 microns to 5 microns.

A cosmetic composition comprising only a small amount of pigments, or even no pigment, but that satisfactorily camouflages the microreliefs and other imperfections of the skin may thus be obtained. According to one embodiment, the at least one first composition of the disclosure comprises pigments in an amount such that they do not reduce the haze index and the transparency index of the composition. The composition may be, for example, free of pigments or nacres.

The mineral filler comprises at least one material chosen, for example, from talc, mica, silica, kaolin, hollow silica microspheres, glass microcapsules, titanium oxides, iron oxides, zinc oxides, cerium oxides, aluminium oxides, barium sulphate, metal hydroxides, and mixtures thereof.

The mineral filler may comprise a mica substrate with a coating chosen from alumina, titanium dioxide, silica, aluminium oxide, and barium sulfate.

The filler. may also be chosen from TiO₂-coated silica composites, silica-coated TiO₂ composites, silica/zinc oxide composites, and mixtures thereof.

The filler may also be in a form chosen from lamellar and non-lamellar forms.

The at least one first composition, for example, comprises a mineral filler comprising mica, the filler may have a mean particle size of less than or equal to 50 microns, for example, less than 10 microns and further, for example, less than 5 microns.

Fillers useful herein may be chosen from, but are not limited to, the following:
silica-coated titanium oxide, such as Flonac TS 40 C distributed by Eckart,
silica microbeads with a particle size ranging from 3 to 12 microns, such as Silica Beads SB 150 manufactured by Miyoshi, or Sunsphere H with a particle size ranging from 3 microns to 12 microns, manufactured by Asahi Glass,
silica platelets, such as Chemiceler, distributed by Sumitomo or Finesil F-80 also distributed by Sumitomo, with a particle size equal to 1.5 microns,
silica coated with titanium dioxide and with porous silica, with a particle size, for example, equal to 0.6 micron, for instance the product ACS-0050510 from Catalysts and Chemicals, such that the silica/titanium dioxide/porous silica proportion is equal to 85/5/10,
mica coated with barium sulfate and with titanium oxide, for instance, the product Naturaleaf from Merck, such that the mica/barium sulfate/titanium oxide proportion is equal to 66/22/12,
a complex of silica and of cerium oxide coated with amorphous silica, with a particle size ranging from 1 microns to 10 microns, for instance, the product Ceriguard SC,
a complex of silica and of titanium oxide coated with polyhydrogenomethylsiloxane, in proportions of 93/5/2,
sericite platelets coated with titanium oxide, alumina and silica, for instance, Coverleaf AR-20121M sold by Catalysts and Chemicals, the particle size ranges from 5 μm to 10 μm and such that the sericite/titanium oxide/alumina/silica proportion is equal to 67/5/18/10, and
talc.

According to some embodiments, the at least one first composition comprises a mineral filler chosen from composites of a mineral silicate and of a metal hydroxide.

The mineral silicate may be, for example, natural or synthetic mica. The mica may be chosen from the muscovite, phlogopite, tiotite, sericite, lepidolite and paragonite micas, synthetic micas, and mixtures thereof.

The metal hydroxide may be a hydroxide chosen from, for example, Ag, Au, Cu, Al, Ni, Sn, Mg, Cr, Mo, Ti, Pt, Va, Rb, W, Zn, Ge, Te, Se, and alloys thereof. For example, aluminium hydroxide may be used.

According to some embodiments, the mineral filler is a composite comprising mineral silicate particles surface-coated with the metal hydroxide. The mineral silicate may be, for example, in a form chosen from lamellar and platelet forms. As used herein, the term “in platelet form” means particles wherein the ratio between the largest dimension and the thickness is greater than or equal to 5, 10, or even 20.

The metal hydroxide may be in a spherical form, for instance, in the form of beads, for example, with a particle size ranging from 1 to 15 microns and further, for example, 10 microns.

The mass proportion between the mineral silicate and the metal hydroxide may range, for example, from 50/50 to 80/20, such as from 60/40 to 70/30 and further, for example, 65/35.

The metal hydroxide beads may be, for example, aluminium hydroxide beads.

Mica platelets coated with aluminium hydroxide microbeads may be used in a weight ratio of 60/40 or 65/35, for example, the product Excel Mica JP-1 or the product Excel Mica JP-2 sold by Miyoshi.

In some embodiments, the at least one first composition may also comprise optionally-modified starch.

The mineral filler is present in an amount ranging from 0.1% to 40%, for example, from 3% to 15%, such as from 2% to 8% and further, for example, 5%, by weight relative to the total weight of the composition.

Examples of the organic fillers of the disclosure include: Nylon® powders, poly-β-alanine powders, polyethylene powders, polytetrafluoroethylene powders, lauroyllysine, starch, hollow polymer microspheres such as those of polyvinylidene chloride/acrylonitrile, for instance Expancel®, acrylic acid copolymers, for instance Polytrap®, and silicone resin microbeads, for example, Tospearl.

Mention may also be made of the of the multilayer structures of polymer compounds.

The at least one first composition, for example, comprises an organic filler comprising starch, the filler having a mean particle size, for example, of less than or equal to 50 microns, for example, less than 10 microns and further, for example, less than 5 microns.

According to some embodiments, the organic filler may be starch, for example, modified starch, for instance the starch crosslinked with octenylsuccinic anhydride sold under the reference Dry Flo Plus (28-1160) by National Starch.

The organic filler may be present in an amount ranging from 0.1% to 40%, for example, from 3% to 15%, such as from 2% to 8% and further, for example, about 5%, by weight relative to the total weight of the composition.

The organic filler and the mineral filler, for example, have a refractive index of greater than 1, such as ranging from 1.25 to 1.9 and further, for example, ranging from 1.45 to 1.55.

The mineral filler and the organic filler are, for example, present in a mass ratio ranging from 1:1 to 1:3.

According to some embodiments, the mineral filler is lamellar when the organic filler is spherical, and alternatively, the mineral filler is spherical when the organic filler is lamellar.

The composition according to the disclosure comprises less than 30% of fillers and, for example, less than 20% of fillers.

According to some aspects, the at least one first composition comprises mica particles surface-treated with aluminium hydroxide, and has
a haze index, (Th−Td)/Th*100, of greater than or equal to 40%, and
a transparency index, Th, of greater than or equal to 70%,
   Th being the hemispheric transmittance of the composition and Td, being the direct transmittance of the composition.

The mica particles may be, for example, present in an amount that is sufficient to obtain a satisfactory haze index and a satisfactory transparency index.

According to further aspects, the at least one first composition comprises coated or uncoated mica particles and modified or unmodified starch particles.

The composition according to the disclosure may further comprise at least one non-volatile oil.

As used herein, the term “non-volatile oil” means a fatty substance that is liquid at room temperature (25° C.) and that does not evaporate at that temperature.

Examples of the non-volatile oils include, but are not limited to, the following:
poly(C₂-C₂₀)alkylsiloxanes and for example, those comprising trimethylsilyl end groups, such as those with a viscosity of less than 0.06 m²/s, e.g., oils of linear polydimethylsiloxanes and alkylmethylpolysiloxanes such as cetyldimethicone (CTFA name);
silicones modified with optional groups chosen from fluorinated aliphatic, aromatic, and functional groups such as, hydroxyl, thiol and/or amine groups;
phenylsilicone oils, for example, phenyl trimethicones;
oils of plant or mineral origin, such as liquid paraffin, liquid petroleum jelly, perhydrosqualene, lanolin, apricot oil, wheatgerm oil, sweet almond oil, beauty-leaf oil, sesame seed oil, macadamia oil, grapeseed oil, rapeseed oil, coconut oil, groundnut oil, sunflower oil, palm oil, castor oil, avocado oil, jojoba oil, olive oil, cereal germ oil, the liquid fraction of shea butter, fatty acid esters of polyols (for example, liquid triglycerides), isopropyl myristate, miglyol oil, isostearyl neopentanoate, 2-ethylhexyl palmitate, castor oil, tributyl acetyl citrate, alcohols (for example 2-octyldodecanol), acetylglycerides; octanoates, decanoates of alcohols and of polyalcohols, ricinoleates of alcohols and of polyalcohols, fatty acid triglycerides, glycerides, and fluoro and perfluoro oils;
amide compounds, for example, those described in Patent Application No. PCT/FR98/01077, for instance, N-neopentanoyl-2-octyldodecylamine, N-neopentanoyl-2-butyloctylamine, N-(3,5,5-trimethylhexanoyl)-2-octyldodecylamine or N-(3,5,5-trimethylhexanoyl)-2-butyloctylamine; and
mixtures thereof.

The non-volatile oil of the fatty phase of the composition according to the disclosure comprises at least one oil chosen from hydrogenated polyisobutene, arachidyl propionate, octyldodecyl neopentanoate, polybutene, dimethicones, octyldodecanol, and mixtures thereof.

The non-volatile oil is present in an amount ranging from 1% to 85% and, for example, from 5% to 50%, by weight relative to the total weight of the composition.

The composition may also comprise a wax and/or a pasty compound, for example, when it is in the form of a stick.

As used herein, the term “pasty compound” means a lipophilic fatty compound with a reversible solid/liquid change of state, that has an anisotropic crystal organization in the solid state, and comprises at a temperature of 23° C. a liquid fraction and a solid fraction. In the present disclosure, the pasty compound, for example, may have a hardness at 20° C. ranging from 0.001 MPa to 0.5 MPa and for example, from 0.002 MPa to 0.4 MPa.

The hardness may be measured by penetrating a probe into a sample of compound and for example, using a texture analyzer (e.g., the TA-XT2i machine from Rheo) equipped with a 2 mm diameter stainless-steel cylinder. The hardness measurement is performed at 20° C. at the center of 5 samples. The cylinder is introduced into each sample at a prespeed of 1 mm/s and then at a measuring speed of 0.1 mm/s, with the penetration depth being 0.3 mm. The maximum peak recorded is the hardness value.

At a temperature of 23° C., this pasty compound may also be in the form of a liquid fraction and a solid fraction, i.e., the starting melting point of the pasty compound may be less than 23° C. The liquid fraction of the pasty compound measured at 23° C. is present in an amount ranging from 9% to 97%, by weight of the compound. This liquid fraction at 23° C., for example, is present in an amount ranging from 15% to 85% and further, for example, from 40% to 85%, by weight.

The liquid fraction by weight of the pasty compound at 23° C. may be equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound may be equal to the area under the curve of a thermogram obtained using a differential scanning calorimeter (DSC), such as the MDSC 2920® calorimeter by the company TA Instrument, with a temperature rise of 5° C. or 10° C. per minute, according to standard ISO 11357-3:1999. The heat of fusion of the pasty compound is the amount of energy required to make the compound change from the solid state to the liquid state and is expressed in units of J/g.

The heat of fusion consumed at 23° C. is the amount of energy absorbed by the sample to change from the solid state to the state that it has at 23° C., comprising a liquid fraction and a solid fraction.

The liquid fraction of the pasty compound, measured at 32° C., for example, is present in an amount ranging from 30% to 100% by weight of the compound, such as from 80% to 100% and further, for example, from 90% to 100%, by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. may be calculated in the same manner as the heat of fusion consumed at 23° C.

The pasty compound may be, for example, chosen from synthetic compounds and compounds of plant origin. A pasty compound may be obtained by synthesis from starting materials of plant origin.

Pasty compounds may be chosen from, but are not limited to, the following:
lanolin and derivatives thereof,
polymer and non-polymer silicone compounds,
polymer and non-polymer fluoro compounds,
vinyl polymers, such as:
   olefin homopolymers,
   olefin copolymers,
   hydrogenated diene homopolymers and copolymers,
   linear or branched oligomers, which are homopolymers or copolymers of alkyl (meth)acrylates optionally comprising a C₈-C₃₀ alkyl group
   oligomers, which are homopolymers and copolymers of vinyl esters comprising C₈-C₃₀ alkyl groups
   oligomers, which are homopolymers and copolymers of vinyl ethers comprising C₈-C₃₀ alkyl groups,
liposoluble polyethers resulting from the polyetherification between at least one C₂-C₁₀₀ such as, C₂-C₅₀ diols,
esters, and
mixtures thereof.

The pasty compound, for example, is present in an amount ranging from 1% to 99%, such as 1% to 60%, further, for example 2% to 30% and even still, for example, 5% to 15%, by weight of the composition.

Examples of the waxes maybe chosen from, but are not limitedto, the following:
waxes of animal, plant, mineral or synthetic origin, such as microcrystalline waxes, paraffin, petrolatum, petroleum jelly, ozokerite or montan wax; beeswax and derivatives thereof; lanolin and derivatives thereof; candelilla wax, ouricury wax, carnauba wax, Japan wax, cocoa butter, cork fibre wax or sugarcane wax; hydrogenated oils that are solid at 25° C., ozokerites, fatty esters and glycerides that are solid at 25° C.; polyethylene waxes and the waxes obtained by Fischer-Tropsch synthesis; hydrogenated oils that are solid at 25° C.; fatty esters that are solid at 25° C.; silicone waxes; fluoro waxes; and mixtures thereof.

The composition according to the disclosure may also contain fillers that do not allow the composition to be given the properties described previously.

These fillers may be present in the at least one first composition in a proportion ranging from 0% to 20%, by weight and, for example, 2% to 15%, by weight relative to the total weight of the composition. In some embodiments, the at least one first composition is free of such fillers

Second Composition

The cosmetic makeup product according to the disclosure comprises at least one second composition comprising a second physiologically acceptable medium.

The at least one second composition may be any composition known to those skilled in the art, of which the person skilled in the art wishes to improve at least one cosmetic property, for example, to improve the staying power, the migration resistance and/or the masking of the imperfections of keratin materials.

When the cosmetic product of the disclosure is a makeup product, the at least one second composition may be any cosmetic makeup composition known to those skilled in the art. For example, the at least one second composition may be a lipstick or a gloss.

According to some embodiments of the disclosure, the physiologically acceptable medium of the at least one second composition comprises a liquid phase that is non-volatile at room temperature and atmospheric pressure.

As used herein, the term “non-volatile liquid phase” means any medium capable of remaining on the skin or the lips for several hours. A non-volatile liquid phase, for example, may have a non-zero vapor pressure at room temperature and atmospheric pressure, of less than 0.02 mmHg (2.66 Pa) and for example, less than 10⁻³ mmHg (0.13 Pa).

The non-volatile liquid phase of the at least one second composition may be chosen from a liquid hydrocarbon-based phase, a liquid silicone phase, and a liquid fluoro phase at room temperature.

According to some embodiments, the at least one second cosmetic composition comprises an agent chosen from nacres, monochromatic coloring, reflective coloring, and goniochromatic coloring agents.

According to some embodiments, the at least one second cosmetic composition comprises, in a physiologically acceptable medium, at least one goniochromatic coloring agent and at least some reflective particles that are distinct from the goniochromatic coloring agent.

The reflective particles may be chosen from, but are not limited to, the following:
particles with a natural and synthetic substrate coated at least partially with at least one layer of at least one metal,
particles with a synthetic substrate coated at least partially with at least one layer of at least one metallic compound and for example, a metal oxide,
particles formed from a stack of at least two layers of materials with different refractive indices, at least one of these layers possibly being a polymer, and
metal oxide particles.

As used herein, the term “goniochromatic coloring agent” means a coloring agent for obtaining, when the cosmetic composition is spread onto a support, a color trajectory in the a*b* plane of the CIE 1976 colorimetric space corresponding to a variation Dh of the hue angle h of at least 20° when the angle of observation relative to the normal is varied ranging from 0° and 80°, for an incident light angle of 45°.

The color trajectory may be measured, for example, using an Instrument Systems brand spectrogonioreflectometer of reference GON 360 Goniometer. After the at least one second composition has been spread in fluid form to a thickness of 300 μm using an automatic spreader onto an Erichsen brand contrast card of reference Typ 24/5, the measurement is performed on the black background of the card.

According to some embodiments, a goniochromatic coloring agent makes it possible to observe a color change, also known as a “color flop”, as a function of the angle of observation, which is greater than the change that may be encountered with nacres.

As used herein, the term “reflective particles” means particles wherein the size, structure, for example, the thickness of the layer(s) of which they are composed, and their physical and chemical nature, and the surface state, allow them to reflect the incident light with an intensity sufficient to be able to create at the surface of the claimed composition, when the composition is applied to the support to be made up, highlight points that are visible to the naked eye, i.e., more luminous points that contrast with their surroundings by appearing to shine.

Reflective particles may cloud the visual perception of the curvature of the made-up support, by tending to prevent long-lasting visual focusing, the highlight points being capable of appearing or disappearing randomly when the made-up support and the observer are in motion.

According to some embodiments, the mean gloss of the at least one second composition may exceed a certain threshold, for example, when the composition is intended to be applied to the lips or the nails.

As used herein, the term “mean gloss” means the gloss as may be conventionally measured using a glossmeter, by the following method.

To measure the mean gloss, a layer 50 μm in thickness of the present composition is spread, using an automatic spreader, onto a Leneta brand contrast card of reference Form 1A Penopac. The layer of the composition covers at least the white background of the card. Measurement of the gloss at 20° is then performed on the white background using a Byk Gardner brand glossmeter of reference micro TRI-GLOSS.

The mean gloss of the at least one second composition may, for example, be greater than or equal to 30, 50, and even, 70, such as when the composition is intended to be applied to the lips.

The composition may comprise a gloss base intended to allow the desired mean gloss to be obtained.

As used herein, the term “base” means the cosmetic composition without the coloring agent(s) that the composition may comprise.

The cosmetic composition may thus comprise, in some embodiments, a base with a mean gloss greater than 20, 50 and further, for example, 70, such as when the composition is intended to be applied to the lips.

The presence of the coloring agents such as the goniochromatic pigments or of the reflective particles in the gloss base may result in a cosmetic composition with a mean gloss that may be identical or different from the mean gloss of the base considered in isolation.

For example, the formulation of the base may thus be different depending on whether the cosmetic composition is intended to form a liquid gloss or a lipstick. A lipstick base with a mean gloss of about 60, a liquid gloss base or an eyeshadow base with a mean gloss of about 70, and a nail varnish base with a mean gloss of about 50 may be chosen, for example.

The cosmetic composition may comprise when the composition is intended to be applied to the lips, for example, an oily phase, such as an oily phase with a refractive index ranging from 1.47 to 1.51 that can allow a relatively high gloss to be obtained.

The reflective particles may be present in the composition in a homogeneously dispersed form, for example, in an amount ranging from 0.1% to 20% relative to the total weight of the composition, for example, from 1% to 15% by weight and further, for example, from 1% to 10% by weight, such as about 2%, when the composition is intended to be applied to the lips.

According to some embodiments, the reflective particles may be introduced such that the reflective particles/goniochromatic pigments weight ratio ranges from 0.3 to 3 and, for example, from 0.5 to 2.5. This ratio may vary as a function of the nature of the cosmetic composition wherein the particles are incorporated. For example, in a formulation of nail varnish type, this reflective particles/goniochromatic pigments weight ratio may be greater than 1, for example, greater than 1.5 and such as, greater than or equal to 2. On the other hand, in formulations of liquid lipstick type or in the form of tubes, this weight ratio may be less than or equal to 2 and for example, less than or equal to 1.5.

The reflective particles may be chosen from goniochromatic and non-goniochromatic particles, and interference and non-interference particles. In at least one embodiment, the reflective particles are non-goniochromatic particles.

The reflective particles may have a size of at least 10 μm, for example ranging from 20 μm to 50 μm.

As used herein, the term “size” means the size given by the statistical particle size distribution to half the population, known as the D50. The size of the reflective particles may depend on their surface state. The more reflective their surface state, the smaller may be the size, in principle, and vice versa.

The reflective particles may be in varied forms. These particles may be in a form chosen from platelets and globular, such as spherical.

As used herein, the term “platelet form” means particles wherein the ratio of the largest size to the thickness is greater than or equal to 5, or for example 10, or further, for example, 20. The thickness of the particles in platelet form, for example, ranges from 0.5 μm to 5 μm.

Particles with a substantially flat outer surface may, for example, be used since they can give rise more readily, if their size, structure and surface state allow it, to an intense specular reflection. This is referred to as a mirror effect.

It may be desirable for the reflective particles to be non-scattering and non-matt.

Whatever the particle shape, the reflective particles may or may not have a multilayer structure, and, in the case of a multilayer structure, for example, at least one layer of uniform thickness, such as a reflective material.

When the reflective particles do not have a multilayer structure, the particles may comprise, for example, metal oxides, such as titanium oxide or iron oxide, obtained synthetically so as to have a substantially flat surface having, for example, a non-matt and non-scattering surface state, allowing a specular reflection of light that is sufficient to obtain highlight points within the cosmetic composition.

When the reflective particles do have a multilayer structure, these particles may comprise, for example, a natural or synthetic substrate, such as a synthetic substrate at least partially coated with at least one layer of a reflective material.

Whatever the shape of the reflective particles, the substrate may, when it is synthetic, be prepared with a shape that promotes the formation of a reflective surface after coating, such as after depositing a layer of reflective material. The substrate may, for example, have a flat surface and the layer of reflective material, a substantially uniform thickness.

An example of reflective particles comprising a mineral substrate coated with a layer of metal may be a borosilicate substrate coated with silver, also known as “white nacres”.

Further examples of reflective particles include, but are not limited to, particles with a glass substrate coated with silver, in the form of platelets, are sold under the name Microglass Metashine REFSX 2025 PS by the company Toyal and particles with a glass substrate coated with nickel/chromium/molybdenum alloy are sold under the name Crystal Star GF 550 and GF 2525 by this same company.

Irrespective of their shape, the reflective particles may also be chosen from particles with a synthetic substrate coated at least partially with at least one layer of at least one metallic compound, such as a metal oxide, chosen, for example, from titanium oxides, such as TiO₂; iron oxides, such as Fe₂O₃; tin oxide; chromium oxide; barium sulphate; and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

Examples of reflective particles further include synthetic mica substrates coated with titanium dioxide, or particles of glass coated either with brown iron oxide or with titanium oxide, tin oxide or a mixture thereof sold under the brand name Reflecks® by the company Engelhard.

Pigments of the Metashine 1080R rangesold by the company Nippon Sheet Glass Co. Ltd., are also suitable for the disclosure. These pigments, described in Japanese Patent Application No. JP 2001-11340, are flakes of C-Glass comprising from 65% to 72% SiO₂, coated with a layer of titanium oxide of rutile type (TiO₂). These glass flakes have a mean thickness of 1 micron and a mean size of 80 microns, i.e. a mean size/mean thickness ratio of 80. They also have blue, green, yellow or silvery glints depending on the thickness of the TiO₂ layer.

Further examples include particles ranging from 80 μm to 100 μm in size, comprising a synthetic mica substrate (e.g., fluorophlogopite) coated with titanium dioxide present in an amount of 12% of the total weight of the particle, sold under the name Prominence by the company Nihon Koken.

The reflective particles may also be chosen from particles formed by a stack of at least two layers with different refractive indices.

These layers may be of polymeric or metallic nature and may, for example, include at least one polymer layer.

Thus, the reflective particles may be particles derived from a multilayer polymer film.

Such particles are described in Patent Application Publication No. WO 99/36477, and U.S. Pat. Nos. 6,299,979 and 6,387,498.

Reflective particles comprising a stack of at least two layers of polymers are sold by the company 3M under the name Mirror Glitter. These particles comprise layers of 2,6-PEN and of polymethyl methacrylate in a mass ratio of 80/20. Such particles are described in U.S. Pat. No. 5,825,643.

The at least one second composition according to the disclosure may, of course, comprise reflective particles of different nature without departing from the context of the present disclosure.

Goniochromatic Coloring Agents

The composition comprises at least one goniochromatic coloring agent to create, when the composition is applied to its support, a colored background whose color changes with the angle of observation and contrasts with the reflective particles. A single goniochromatic coloring agent may be used for ease of implementation.

The goniochromatic coloring agent may be present, for example, in an amount ranging, on a weight basis relative to the total weight of the composition, from 0.1% to 20% or from 2% to 15% and for example, from 2% to 10%, such as when the composition is intended to be applied to the lips. In the case of such a composition, satisfactory results have been obtained for a content of goniochromatic coloring agent ranging from 2% to 8% combined with a content of reflective particles ranging from 1% to 5%, by weight. A nail varnish composition may comprise, for example, from 0.1% to 5% of goniochromatic coloring agent; a foundation may comprise from 10% to 15% thereof; and a lipstick may comprise from 2% to 8% thereof by weight.

The goniochromatic coloring agent may be chosen so as to present a relatively large color change with the angle of observation.

The goniochromatic coloring agent may, thus, be chosen such that a color difference, ΔE, of the cosmetic composition, measured in the CIE 1976 colorimetric space, of at least 2 may be observed for a variation of the angle of observation ranging from 0° to 80° under illumination at 45°.

The goniochromatic coloring agent may also be chosen such that a variation, Dh, of the hue angle of the cosmetic composition, in the CIE 1976 plane, of at least 30° or even at least 40° or at least 60°, or even at least 100°, may be observed for an illumination at 45° and a variation of the angle of observation ranging from 0° to 80°.

The goniochromatic coloring agent may be chosen, for example, from multilayer interference structures and liquid-crystal coloring agents.

In the case of a multilayer structure, the goniochromatic coloring agent may comprise, for example, at least two layers, each layer, which may be different or identical to the other layer(s), being made, for example, from at least one material chosen from the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers, and combinations thereof.

The multilayer structure may or may not have, relative to a central layer, symmetry in the chemical nature of the stacked layers.

Examples of symmetrical multilayer interference structures that may be used in the compositions prepared in accordance with the disclosure are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; and TiO₂/SiO₂/TiO₂ and TiO₂/Al₂O₃/TiO₂, pigments having these structures being sold under the name Xirona by the company Merck (Darmstadt). A further example of symmetrical multilayer interference structures may be Infinite Colors pigments from the company Shiseido. Depending on the thickness and the nature of the various layers, different effects are obtained. Thus, with the Fe₂O₃/SiO₂/Al/SiO₂/Fe₂O₃ structure, the color changes from green-golden to red-grey for SiO₂ layers ranging from 320 nm to 350 nm; from red to golden for SiO₂ layers ranging from 380 nm to 400 nm; from violet to green for SiO₂ layers ranging from 410 nm to 420 nm; and from copper to red for SiO₂ layers ranging from 430 nm to 440 nm.

Goniochromatic coloring agents that are also suitable for the disclosure are pigments of silica/titanium oxide/tin oxide structure sold under the name Xirona Magic by the company Merck, pigments of silica/zinc oxide structure sold under the name Xirona Indian Summer by the company Merck, and pigments of silica/titanium oxide/mica/tin oxide structure sold under the name Xirona Caribbean Blue by the company Merck.

Goniochromatic coloring agents with a multilayer structure comprising an alternation of polymer layers, for example, of the type such as polyethylene naphthalate and polyethylene terephthalate, may also be used. Such agents are described especially in Patent Application Publication Nos. WO-A-96/19347 and WO-A-99/36478.

Examples of liquid-crystal goniochromatic particles that may be used include, for example, those sold by the company Chenix and also the products sold under the name Helicone® HC by the company Wacker.

The composition may also comprise dispersed goniochromatic fibers. Such fibers may, for example, have a size ranging from 200 μm to 700 μm, such as 300 μm.

For example, interference fibers with a multilayer structure may be used. Fibers with a multilayer structure of polymers are described in European Patent Nos. EP-A-921 217, EP-A-686 858, and U.S. Pat. No. 5,472,798. The multilayer structure may comprise at least two layers, each layer, which may be identical or different, being made of at least one synthetic polymer. The polymers present in the fibers may have a refractive index ranging from 1.30 to 1.82 and, for example, ranging from 1.35 to 1.75. The polymers that may be used include: polyesters such as, polyethylene terephthalate; polyethylene naphthalate; polycarbonate; acrylic polymers such as polymethyl methacrylate; and polyamides.

Goniochromatic fibers with a polyethylene terephthalate/nylon-6 two-layer structure are sold by the company Teijin under the name Morphotex.

Gloss Base

The at least one second composition may also comprise at least one compound capable of giving it gloss, and for example, an oily phase, such as an oily phase with a refractive index ranging from 1.47 to 1.51 such as ranging from 1.48 to 1.50.

The refractive index is measured at room temperature (25° C.) using a refractometer. Such an oily phase may prove to be useful, e.g., in the case of a liquid gloss.

According to some embodiments of the disclosure, the gloss base selected may be one as described in European Patent Application No. EP-A-792 637, the entire disclosure and subject matter of which is hereby incorporated herein by reference.

The cosmetic composition may comprise, for example, at least one oil chosen from carbon-based oil, hydrocarbon-based oil, and fluoro oil and silicone oil of mineral, plant and synthetic origin.

Additives

The at least one first and the at least one second composition in the cosmetic product according to the disclosure may also contain at least one cosmetic, dermatological, hygiene or pharmaceutical active agent.

The at least one cosmetic, dermatological, hygiene or pharmaceutical active agent that may be used in the compositions according to the disclosure may include moisturizers (polyols, i.e., glycerol), vitamins (C, A, E, F, B or PP), essential fatty acids, essential oils, ceramides, sphingolipids, liposoluble sunscreens or sunscreens in the form of nanoparticles, and specific skin-treatment active agents (i.e., protective agents, antibacterial agents, antiwrinkle agents, etc.).

These active agents are used in an amount that is usual for a person skilled in the art and, for example, may be present in an amount ranging from 0% to 20% and such as from 0.001% to 15%, relative to the total weight of the at least one first and the at least one second composition.

Each composition in the product according to the disclosure may further comprise, depending on the intended type of application, constituents conventionally used in the fields under consideration, which are present in an amount that is suitable for the desired presentation form.

In general, the physiologically acceptable media for each of the at least one first and/or the at least one second composition in the product according to the disclosure may comprise, in addition to the oils, additional fatty substances that may be chosen from waxes, oils, gums, and pasty fatty substances.

For example, the physiologically acceptable medium for the at least one first and/or the at least one second composition comprises a pasty fatty substance and/or a wax chosen from the waxes mentioned above.

Each composition of the product according to the disclosure may also comprise any other additive usually used in such compositions, for instance, oil thickeners, aqueous-phase thickeners (e.g., acrylic gelling agent), antioxidants, fragrances, preserving agents (e.g., pentylene glycol), surfactants, or liposoluble polymers (e.g., polyvinylpyrrolidone/eicosene copolymer).

When the physiologically acceptable medium for the at least one first and/or the at least one second composition comprises a liquid organic phase, this medium may, for example, comprise water dispersed or emulsified in the liquid organic phase.

In some embodiments of the disclosure, the compositions can be prepared in the usual manner by a person skilled in the art. The compositions may be in the form of a cast product such as, in the form of a stick or tube, in the form of a soft paste in a heating bag or in the form of a dish which can be used by direct contact or with a sponge. For example, the compositions may be in the form of a cast foundation, a cast makeup rouge or eye shadow, for example, colored, a lipstick, a lip gloss or a concealer product. The composition may also be in the form of a soft paste or alternatively, in the form of a gel or a more or less fluid cream. In this case, the composition may comprise fluid or pasty foundations or lipsticks, lip glosses, antisun products or skin-coloring products, eyeliner or body makeup products, or alternatively, the compositions may have care properties and may then be in the form of a base or of a lipcare balm.

Each composition in the product according to the disclosure may be in any presentation form normally used for topical application such as, in the form of an oily or aqueous solution, an oily or aqueous gel, an oil-in-water or water-in-oil emulsion, a multiple emulsion or a dispersion of oil in water by means of vesicles, the vesicles being located at the oil/water interface, or a powder. Each composition may be fluid or solid.

For example, the at least one first or second composition, or both of them, may have a continuous fatty phase and may, for example, be in anhydrous form and may comprise less than 5% water, and further, for example less than 1% water, relative to the total weight of the at least one first or second composition. The whole two-coat makeup product may be in an anhydrous form.

Each first and second composition may have the appearance of a lotion, a cream, an ointment, a soft paste, a salve, a cast, or molded solid, which is, for example, in stick or dish form, or alternatively a compacted solid.

Each composition may be packaged separately in the same packaging article, for example, in a two-compartment pen, the base composition being delivered from one end of the pen and the top composition being delivered from the other end of the pen, each end being closed such as, in a leaktight manner, by a cap.

The composition may also be applied as a first coat in solid form, thus may allow a more practical application, better stability over time and at elevated temperature of the composition, and may allow the makeup to be applied in a precise line, which is highly desirable in the case of a lipstick or an eyeliner.

The product according to the disclosure may, for example, be used for making up the skin and/or the lips and/or integuments depending on the nature of the ingredients used. For example, the product of the disclosure may be in the form of a solid foundation, a tube of lipstick or lipstick.paste, a concealer product or products for the contours of the eyes, an eyeliner, a mascara, an eyeshadow, a nail varnish, a body makeup product or a skin coloring product.

The product may be used in the form of a lipstick.

For example, the at least one first and/or second composition may be in solid form.

In some embodiments, the topcoat may have care and/or gloss properties.

Further aspects of the disclosure may be a lipstick product, a foundation, a tattoo, a makeup rouge, or an eyeshadow comprising at least one first and at least one second composition, as described above.

The compositions of the disclosure may be obtained by heating the various constituents to the melting point of the highest-melting waxes, followed by casting the mixture in a mold (dish or finger stall). They may also be obtained by extrusion, as described in patent application European Patent Application EP-A-0 667 146.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples below serve to illustrate the present disclosure in a non-limiting manner.

The amounts are given as mass percentages.

### EXAMPLE 1

Smoothing Composition for the Lips

| | |
|---|---|
| Hydrogenated polyisobutene (Nof Corp.) | 10% |
| Arachidyl propionate (Alzo) | 1.8% |
| Octyldodecyl neopentanoate (Bernel) | 1.3% |
| Disteardimonium hectorite (Elementis) | 0.3% |
| Mica coated with aluminium hydroxide microbeads (Excel Mica | 5% |
| JP-2 ® from Miyoshi) | |
| Starch crosslinked with octenylsuccinic anhydride (Dry Flo Plus | 10% |
| (28-1160) ® from National Starch) | |
| Polybutene | 4.9% |
| Polyethylene wax | 15.8% |
| Dimethicone (DC 200-ScSt) | 17.3% |
| Decamethyltetrasiloxane (DC 200-1.5 cSt) | 22% |
| Octyldodecanol | 3.2% |
| Oxypropylenated lanolin wax (5 PO) (Cognis) | 8.4% |

### EXAMPLE 2

Liquid Gloss Lipstick

| | |
|---|---|
| Poly(bis-diglyceryl 2-acryladipate) | 17.5% |
| Diisostearyl malate | 9.5% |
| Tridecyl trimellitate | 10% |
| C18-36 acid triglyceride | 19% |
| Dimethyl silylate silica | 8% |
| Silver-coated glass particles | 2% |
| (Metashine ®)* | |
| Goniochromatic pigment (Sicopearl ®)** | 5% |
| Nacre | 3% |
| Polybutene | 12% |
| Pentaerythrityl tetraisostearate | 13% |
| Fragrance, preserving agent | qs |

*sold by the company Toyal
**sold by the company BASF

The liquid gloss lipstick may be applied over the smoothing composition of Example 1.

1. A cosmetic makeup and/or care product comprising at least one first and at least one second composition,
the at least one first composition comprising a first physiologically acceptable medium and having a haze index, (Th−Td)/Th*100, of greater than or equal to 40%, and a transparency index, Th, of greater than or equal to 70%, and
the at least one second composition comprising a second physiologically acceptable medium.

2. The product according to claim 1, wherein the haze index is greater than or equal to 50%.

3. The product according to claim 1, wherein the transparency index is greater than or equal to 85%.

4. The product according to claim 3, wherein the transparency index is greater than or equal to 95%.

5. The product according to claim 1, wherein the at least one first composition further comprises at least one compound chosen from mineral and organic compounds and is present in the composition in an amount that is sufficient for the at least one first composition to have a haze index of greater than or equal to 40% and a transparency index of greater than or equal to 70%.

6. The product according to claim 1, wherein the at least one first composition comprises at least one compound chosen from fillers, organopolysiloxanes, and non-silicone polymers of low molecular mass.

7. The product according to claim 6, wherein the fillers have a mean particle size less than or equal to 50 microns.

8. The product according to claim 7, wherein the fillers have a mean particle size less than or equal to 30 microns.

9. The product according to claim 8, wherein the fillers have a mean particle size less than or equal to 15 microns.

10. The product according to claim 9, wherein the fillers have a mean particle size ranging from 3 to 5 microns.

11. The product according to claim 6, wherein the fillers are chosen from fillers of natural and synthetic origin.

12. The product according to claim 11, wherein the fillers of natural and synthetic origin are chosen from talc, starch, mica, silica, Nylon powders, polyethylene powders, poly-β-alanine, poly(methyl methacrylates), and mixtures thereof.

13. The product according to claim 6, wherein the organopolysiloxanes are chosen from:
i) polyorganopolysiloxanes comprising at least one unit chosen from R₂SiO and RSiO_{1.5} and optionally at least one unit chosen from R₃SiO_{0.5} and SiO₂, wherein R, R₂ and R₃, which may be identical or different, are chosen from hydrogen atoms, alkyl, aryl and unsaturated aliphatic groups, and wherein the weight ratio of R₂SiO to RSiO_{1.5} ranges from 1:1 to 30:1;
ii) polyorganopolysiloxanes that are insoluble and swellable in silicone oil, obtained by addition of an organohydrogenopolysiloxane (1) and an organopolysiloxane (2) comprising unsaturated aliphatic groups, such that the amount of hydrogen or of unsaturated aliphatic groups in (1) and (2), respectively, ranges from 1 mol % to 20 mol % when the organopolysiloxane is non-cyclic and ranges from 1 mol % to 50 mol % when the organopolysiloxane is cyclic.

14. The product according to claim 13, wherein R, R₂ and R₃, which are identical or different, are alkyl groups chosen from methyl, ethyl, and propyl.

15. The product according to claim 13, wherein R, R₂ and R₃, which are identical or different, are aryl groups chosen from phenyl and tolyl.

16. The product according to claim 13, wherein R, R₂ and R₃, which are identical or different, are aliphatic groups chosen from vinyl.

17. The product according to claim 6, wherein the non-silicone polymers of low molecular mass are chosen from amide, urethane, and urea polymers with a weight-average molecular mass ranging from 200 to 1000 g/mol.

18. The product according to claim 17, wherein the polymers are chosen from pendent and terminal alkyl and alkylene chains comprising from 8 to 60 carbon atoms.

19. The product according to claim 18, wherein the polymers are chosen from pendent and terminal alkyl and alkylene chains comprising 12 to 40 carbon atoms.

20. The product according to claim 1, wherein the at least one first composition comprises a mineral filler and an organic filler.

21. The product according to claim 20, wherein the mineral filler is lamellar when the organic filler is spherical, and the mineral filler is spherical when the organic filler is lamellar.

22. The product according to claim 20, wherein the mineral filler comprises at least one material chosen from talc, mica, silica, kaolin, hollow silica microspheres, glass microcapsules, titanium oxides, iron oxides, zinc oxides, aluminium oxides, barium sulphate, metal hydroxides, and mixtures thereof.

23. The product according to claim 20, wherein the organic filler is chosen from polyethylene powder, starch powder, modified starch powder, Nylon powder, styrene/acrylic copolymer powders, and mixtures thereof.

24. The product according to claim 23, wherein the organic filler comprises starch crosslinked with octenylsuccinic anhydride.

25. The product according to claim 1, wherein the at least one first composition comprises a mineral filler comprising mica and having a mean particle size of less than or equal to 50 microns.

26. The product according to claim 25, wherein the mineral filler has a mean particle size less than 10 microns.

27. The product according to claim 25, wherein the mineral filler has a mean particle size less than 5 microns.

28. The product according to claim 1, wherein the at least one first composition comprises an organic filler comprising starch and having a mean particle size of less than or equal to 50 microns.

29. The product according to claim 28, wherein the organic filler has a mean particle size less than 10 microns.

30. The product according to claim 29, wherein the organic filler has a mean particle size less than 5 microns.

31. The product according to claim 6, wherein the at least one compound is present in an amount ranging from 0.1% to 40%, by weight relative to the total weight of the composition.

32. The product according to claim 31, wherein the at least one compound is present in an amount ranging from 3% to 15% by weight relative to the total weight of the composition.

33. The product according to claim 1, wherein the at least one first composition comprises a volatile oil.

34. The product according to claim 1, wherein the at least one second composition has a mean gloss greater than or equal to 30.

35. The product according to claim 34, wherein the at least one second composition has a mean gloss greater than or equal to 50.

36. The product according to claim 35, wherein the at least one second composition has a mean gloss greater than or equal to 70.

37. The product according to claim 34, wherein the at least one second composition comprises, in a physiologically acceptable medium, at least one additional ingredient chosen from goniochromatic coloring agents and reflective particles.

38. The product according to claim 1, wherein the at least one first or at least one second composition, or both, is in a form chosen from a foundation, a makeup rouge, an eyeshadow, a lipstick, a product with care properties, an eyeliner, a concealer product, and a body makeup product.

39. The product according to claim 1, wherein the at least one first or at least one second composition, or both, is in anhydrous form.

40. The product according to claim 1, wherein the at least one second composition is in a form chosen from a lipstick in tube and a liquid gloss.

41. A process for making up the skin, the lips and/or integuments comprising
applying to the skin, the lips and/or integuments, in any order:
a coat of at least one first composition comprising a first physiologically acceptable medium and comprising at least one filler that gives the at least one first composition a haze index, (Th−Td)/Th*100, of greater than or equal to 40% and a transparency index, Th, of greater than or equal to 70%, and
a coat of at least one second composition comprising a second physiologically acceptable medium.

42. A makeup kit comprising at least two separate compartments,
wherein a first compartment comprises at least one first composition comprising a first physiologically acceptable medium and having a haze index, (Th−Td)/Th*100, of greater than or equal to 40%, and a transparency index, Th, of greater than or equal to 70%, and
wherein a second compartment comprises at least one second composition comprising a second physiologically acceptable medium.

43. A process for modifying the appearance of the surface of a body part chosen from the skin, lips and/or integuments and/or for modifying the perception of the volume of said body part comprising
applying to said body part a cosmetic make-up and/or care product comprising:
at least one first composition comprising a first physiologically acceptable medium and having a haze index, (Th−Td)/Th*100, of greater than or equal to 40%, and a transparency index, Th, of greater than or equal to 70%, and
at least one second composition comprising a second physiologically acceptable medium, and
obtaining a glossy deposit that has staying power, does not migrate, and maintains its color and gloss.
